(19) United States
(12) Patent Application Publication
(10) Pub. No.: US 2026/0272834 A1
(43) Pub. Date: Sep. 17, 2026

(54) EXTRACELLULAR VESICLE, PREPARATION METHOD, PHARMACEUTICAL COMPOSITION AND APPLICATION THEREOF

(71) Applicant: SHENZHEN INSTITUTES OF ADVANCED TECHNOLOGY CHINESE ACADEMY OF SCIENCES, SHENZHEN (CN)

(72) Inventors: HUI YANG, SHENZHEN (CN); ZITONG YU, SHENZHEN (CN); YI ZHANG, SHENZHEN (CN); SIHUI CHEN, SHENZHEN (CN)

(21) Appl. No.: 19/666,386

(22) Filed: May 4, 2026

Related U.S. Application Data
(63) Continuation of application No. PCT/CN2024/082432, filed on Mar. 19, 2024.

(30) Foreign Application Priority Data
Nov. 6, 2023 (CN) 202311467315.2

(51) Int. Cl. A61K 9/127; A61K 9/51; A61K 31/7088; B82Y 5/00; C12Q 1/6851; C12Q 1/686

(52) U.S. Cl. CPC A61K 9/127; A61K 9/5184; A61K 9/5192; A61K 31/7088; C12Q 1/6851; C12Q 1/686; B82Y 5/00

(57) ABSTRACT

The disclosure provides an extracellular vesicle, a preparation method, a pharmaceutical composition, and application thereof. The preparation method includes generating squeezing effects on extracellular vesicles, to load exogenous nucleic acids into the extracellular vesicles; purifying the extracellular vesicles loaded with the exogenous nucleic acids, to obtain the extracellular vesicles with high levels of the exogenous nucleic acids for diagnosis and treatment; and quantifying the exogenous nucleic acids of the extracellular vesicles with high levels of the exogenous nucleic acids for diagnosis and treatment. The disclosure loads the exogenous nucleic acids having diagnosis and treatment properties into the extracellular vesicles by a high-throughput physical squeezing method.

### CROSS-REFERENCE TO RELATED APPLICATIONS

This application is a continuation of co-pending International Patent Application Number PCT/CN2024/082432, filed on Mar. 19, 2024. The disclosure of which is incorporated herein by reference in its entirety.

### TECHNICAL FIELD

The disclosure relates to biotechnology detection technology field, and particularly to an extracellular vesicle, a preparation method, a pharmaceutical composition, and an application thereof.

### BACKGROUND

Gene therapy is an emerging clinical medical technology born from a combination of modern medicine and molecular biology, that introduces a target gene into a target cell through molecular biology techniques to correct erroneous gene expressions in the target cell, thereby treating diseases. However, nucleic acids are unstable in vitro and easily degraded in vivo, thus assistance of carrier technology is required. A desired carrier should have characteristics such as stability, low immunogenicity, and tissue specificity. The enclosed internal space of extracellular vesicles accommodates loading of various molecules. A phospholipid bilayer provides a protection for the exogenous nucleic acids. A specific molecular distribution ensures a low immunogenicity.

In prior art, high-throughput physical squeezing methods are used to load small molecule compounds for diagnosis and treatment into the extracellular vesicles. However, cell loading achieves endogenous nucleic acid expression through uncontrollable and unquantifiable molecular biology methods. In addition, electroporation methods are used to load exogenous nucleic acids into extracellular vesicles. However, that negatively affects an in-vitro stability of the nucleic acids, thereby negatively affecting a functionality thereof.

### SUMMARY

In view of this, there is necessary to provide extracellular vesicles with high levels of exogenous nucleic acids for diagnosis and treatment, a rapid preparation method, a pharmaceutical composition and an application thereof, for defects of the prior art, with a controllable loading process and a higher in-vitro stability of the nucleic acids.

To resolve the foregoing problem, the present disclosure adopts the following technical solutions:

A first purpose of the present disclosure is to provide a method for preparing extracellular vesicles, comprising the following steps.

Generating squeezing effects on extracellular vesicles, to load exogenous nucleic acids into the extracellular vesicles, wherein a concentration of the extracellular vesicles is 1×10²-1×10²⁰ particles/mL, a squeezing duration is from about 30 seconds to 15 minutes.

Purifying the extracellular vesicles loaded with the exogenous nucleic acids, to obtain the extracellular vesicles with high levels of the exogenous nucleic acids for diagnosis and treatment.

Quantifying the exogenous nucleic acids of the extracellular vesicles with high levels of the exogenous nucleic acids for diagnosis and treatment.

In some embodiments, the step of generating squeezing effects on extracellular vesicles, to load exogenous nucleic acids into the extracellular vesicles, includes the following steps.

Mixing the exogenous nucleic acids with the extracellular vesicles, and then generating the squeezing effects on the extracellular vesicles via geometric constriction within a nanoporation device (ExoNP), inducing transient membrane poration of each of the extracellular vesicles, thereby loading the exogenous nucleic acids into the extracellular vesicles.

In some embodiments, a size of each of the extracellular vesicles is 20 nm-12 μm, and each of the exogenous nucleic acids includes from 4 to 30000 nucleotides.

In some embodiments, the size of each of the extracellular vesicles is 30 nm-200 nm, and each of the exogenous nucleic acids include from 10 to 50 nucleotides.

In some embodiments, a concentration of the exogenous nucleic acids is 10 pM-1 μM, a concentration of the extracellular vesicles is 1×10⁴-1×10¹⁸ particles/mL, and a volume ratio of the exogenous nucleic acids to the extracellular vesicles is 1:50-1:500.

In some embodiments, the concentration of the exogenous nucleic acids is 1 nM-1 μM, the concentration of the extracellular vesicles is 1×10⁴-1×10¹⁸ particles/mL, and the volume ratio of the exogenous nucleic acids to the extracellular vesicles is 1:100-1:200.

In some embodiments, the step of purifying the extracellular vesicles loaded with the exogenous nucleic acids, to obtain the extracellular vesicles with high levels of the exogenous nucleic acids for diagnosis and treatment, includes the following steps.

Purifying the extracellular vesicles loaded with the exogenous nucleic acids by using at least one of an ultrafiltration method and an RNase method to remove free nucleic acids, thereby obtaining the extracellular vesicles with high levels of the exogenous nucleic acids for diagnosis and treatment.

In some embodiments, the step of quantifying the exogenous nucleic acids of the extracellular vesicles with high levels of the exogenous nucleic acids for diagnosis and treatment, includes the following steps.

Quantifying the exogenous nucleic acids of the extracellular vesicles with high levels of the exogenous nucleic acids for diagnosis and treatment, by at least one of real-time quantitative polymerase chain reaction and droplet digital polymerase chain reaction.

In some embodiments, the extracellular vesicles include liposomes, artificial lipid vesicles, lipid nanoparticles, plant extracellular vesicles, animal extracellular vesicles, human cell-derived extracellular vesicles, bacterial outer membrane vesicles, microbial secreted membrane vesicles, cell membrane reassembled nanoparticles, bacterial outer membrane reassembled nanoparticles, enveloped virus particles and virus-like particles, and artificial nanovesicles hybridized with cell membrane and/or bacterial outer membrane and/or viral membrane proteins; the exogenous nucleic acids for diagnosis and treatment include plasmids, ribonucleic acids, deoxyribonucleic acids, oligonucleotides, circular RNA, miRNA, mRNA, antisense oligonucleotides (ASO), siRNA, saRNA, splice-switching oligonucleotides (SSO), and expressed oligonucleotides (EON).

A second purpose of the present disclosure is to provide an extracellular vesicle prepared by the method for rapidly preparing the extracellular vesicles with high levels of the exogenous nucleic acids for diagnosis and treatment.

A third purpose of the present disclosure is to provide a pharmaceutical composition comprising the extracellular vesicles.

A fourth purpose of the present disclosure is to provide for the use of the extracellular vesicles in one of regulating protein expression, protein replacement therapy, immunodiagnosis and immunotherapy, or tissue regeneration.

The present disclosure adopts the above-mentioned technical solutions, and the beneficial effects are as follows:

The present disclosure provides an extracellular vesicle, a preparation method, a pharmaceutical composition and an application thereof. A squeezing effect is generated on an extracellular vesicle to load exogenous nucleic acids into the extracellular vesicles. The extracellular vesicles loaded with the exogenous nucleic acids is purified to obtain the extracellular vesicles with high levels of the exogenous nucleic acids for diagnosis and treatment. The exogenous nucleic acids of the extracellular vesicles with high levels of the exogenous nucleic acids for diagnosis and treatment are quantified. The present disclosure loads the exogenous nucleic acids for diagnosis and treatment into the extracellular vesicles based on a high-throughput physical squeezing method. By utilizing geometric constriction with a dimension close to or slightly smaller than that of the extracellular vesicles, a squeezing effect is induced on the vesicles, causing the membrane of the extracellular vesicles to undergo transient poration, thereby facilitating the loading the exogenous nucleic acids therein, and protecting the in-vitro stability of the nucleic acids. Furthermore, by quantitatively designing the exogenous nucleic acids of the extracellular vesicles with high levels of the exogenous nucleic acids for diagnosis and treatment, complicated and uncontrollable biochemical processes are avoided. The quantitative exogenous nucleic acids enter target cells and take effect to precisely regulate protein expression, thereby increasing a utilization rate of the exogenous nucleic acids.

### BRIEF DESCRIPTION OF DRAWINGS

It will be appreciated that, for simplicity and clarity of illustration of the technical solutions in the embodiments of the disclosure, the drawings required for the description of the embodiments or the prior art are briefly introduced hereinafter. Obviously, the drawings described below are only some embodiments of the present disclosure, and for a person of ordinary skill in the art, other drawings can be obtained from these drawings without creative efforts.

FIG. 1 is a flow chart of a method for rapidly preparing extracellular vesicles with high levels of the exogenous nucleic acids for diagnosis and treatment, according to an embodiment of the disclosure.

FIG. 2 is a schematic diagram of a working principle of a nanoporation device (ExoNP), according to an embodiment of the disclosure.

FIG. 3 is a graph showing a result of relative expression levels of nucleic acids of extracellular vesicles of different treatment groups, according to an embodiment of the disclosure.

FIG. 4 is a graph showing a result of regulation of target genes in target cells by different treatment groups, according to an embodiment of the disclosure.

FIG. 5 is a graph showing a result of relative expression levels of target genes in target cells of different treatment groups, according to an embodiment of the disclosure.

### DESCRIPTION OF EMBODIMENTS

The embodiments of the disclosure are described in detail hereinafter. Examples of the embodiments are shown in the drawings, wherein the same or similar reference numerals throughout indicate the same or similar elements or elements having the same or similar functions. The embodiments described below with reference to the drawings are exemplary and are intended to explain the disclosure and should not be construed as limiting the disclosure.

In the description of the present disclosure, it is to be understood that, the terms such as “upper”, “lower”, “horizontal”, “inner”, “outer” indicate orientations or positional relationships based on the orientations or positional relationships shown in the drawings. These are only for convenience in describing the disclosure and simplifying the description, and do not indicate or imply that the device or element referred to must have a specific orientation, be constructed and operate in a specific orientation, and therefore cannot be construed as limiting the disclosure.

Furthermore, the terms “first”, “second” are used for descriptive purposes only and cannot be understood as indicating or implying relative importance or implicitly specifying the number of technical features indicated. Thus, a feature defined with “first”, “second” may explicitly or implicitly include one or more of those features. In the description of the disclosure, “plurality” means two or more, unless otherwise expressly and specifically defined.

To make the purpose, technical solutions and advantages of the disclosure clearer, the disclosure is further described in detail below with reference to the drawings and embodiments.

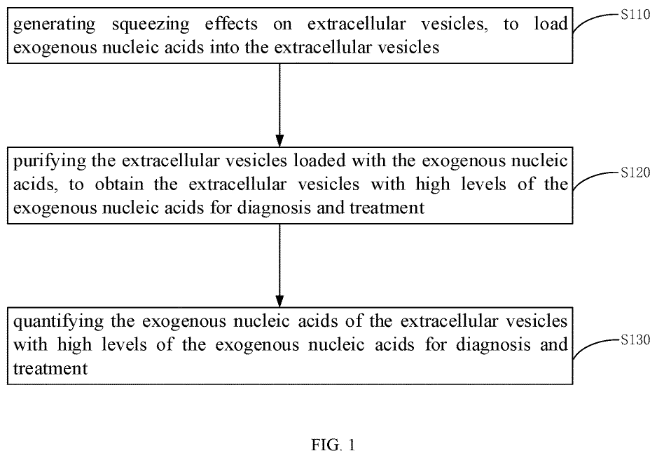

FIG. 1 is a flow chart of a method for preparing extracellular vesicles, according to an embodiment of the present disclosure, that includes the following steps S110 to S130. The implementation of each step is described in detail below.

S110, generating squeezing effects on extracellular vesicles, to load exogenous nucleic acids into the extracellular vesicles. A concentration of the extracellular vesicles is 1×10²-1×10²⁰ particles/mL.

In this embodiment, the step of generating squeezing effects on extracellular vesicles, to load exogenous nucleic acids into the extracellular vesicles, includes the following steps: mixing the exogenous nucleic acids with the extracellular vesicles, and then generating the squeezing effects on the extracellular vesicles via geometric constriction within a nanoporation device (ExoNP), inducing transient membrane poration of each of the extracellular vesicles, thereby loading the exogenous nucleic acids into the extracellular vesicles.

It can be understood that, the extracellular vesicles provided in this embodiment is obtained by conventional isolation steps, including cell recovery, cell culture, isolation and purification of the extracellular vesicles.

The extracellular vesicles include liposomes, artificial lipid vesicles, lipid nanoparticles, plant extracellular vesicles, animal extracellular vesicles, human cell-derived extracellular vesicles, bacterial outer membrane vesicles, microbial secreted membrane vesicles, cell membrane reassembled nanoparticles, bacterial outer membrane reassembled nanoparticles, enveloped virus particles and virus-like particles, and artificial nanovesicles hybridized with cell membrane and/or bacterial outer membrane and/or viral membrane proteins. The exogenous nucleic acids for diagnosis and treatment includes plasmids, ribonucleic acids, deoxyribonucleic acids, oligonucleotides, circular RNA, miRNA, mRNA, antisense oligonucleotides (ASO), siRNA, saRNA, splice-switching oligonucleotides (SSO) and expressed oligonucleotides (EON).

Specifically, a size of each of the extracellular vesicles is 20 nm-12 μm, and each of the exogenous nucleic acids includes from 4 to 30000 nucleotides.

Specifically, the size of each of the extracellular vesicles is 30 nm-200 nm, and each of the exogenous nucleic acids include from 10 to 50 nucleotides.

It can be understood that, the extracellular vesicles smaller than 200 nm can easily cross multiple physiological and pathological barriers in living organisms. Currently, nucleic acid drugs with therapeutic and diagnostic effects mainly include antisense oligonucleotides (ASO, composed of about 15-25 nucleotides), small interfering RNA (siRNA, composed of about 20-25 nucleotides) and microRNA (miRNA, composed of about 21-23 nucleotides).

It can be understood that, the exogenous nucleic acids provided in this embodiment, are obtained by conventional in-vitro synthesis of desired nucleic acids.

Specifically, a concentration of the exogenous nucleic acids is 10 pM-10 μM, a concentration of the extracellular vesicles is 1×10⁴-1×10¹⁸ particles/mL, and a volume ratio of the exogenous nucleic acids to the extracellular vesicles is 1:50-1:500.

Specifically, the concentration of the exogenous nucleic acids is 1 nM-1 μM, and the volume ratio of the exogenous nucleic acids to the extracellular vesicles is 1:100-1:200.

It can be understood that, large-scale purification methods for extracellular vesicles smaller than 200 nm have certain challenges, and current methods can achieve a concentration of 1×10⁶-1×10¹⁶ particles/mL. To achieve a minimum dose of the exogenous nucleic acids for diagnosis and treatment to enter target cells and exert functions thereof, a low concentration of the therapeutic and diagnostic exogenous nucleic acids is loaded into a low concentration of extracellular vesicles to observe whether diagnosis and treatment effects are exerted.

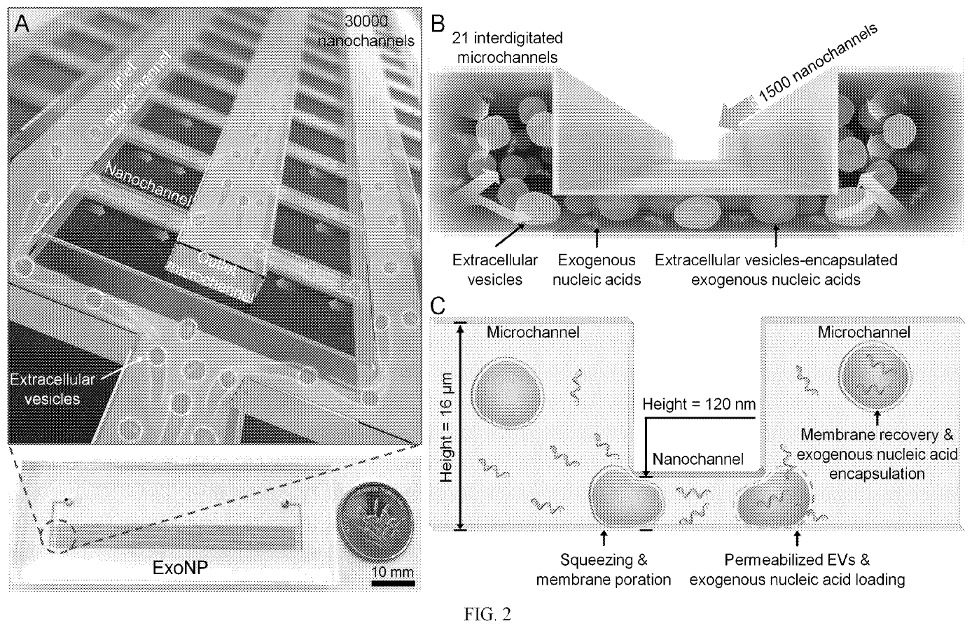

FIG. 2 is a schematic diagram of the working principle of a nanoporation device (ExoNP), according to an embodiment of the present disclosure. This embodiment uses a high-throughput physical squeezing method to load the therapeutic and diagnostic exogenous nucleic acids into the extracellular vesicles. The high-throughput physical squeezing method is based on a micro-nanofluidic technology. Due to the geometric constriction having a dimension close to or slightly smaller than that of the extracellular vesicles, squeezing effects are generated on the extracellular vesicles, causing the membranes of the extracellular vesicles to undergo transient poration, thereby facilitating the loading the exogenous nucleic acids therein, and protecting the in-vitro stability of the exogenous nucleic acids.

It should be noted that, the method provided in the above embodiments is suitable for loading different nucleic acid mixtures, and a time required to process samples with different concentrations is about 10 seconds to 15 minutes.

S102, purifying the extracellular vesicles loaded with the exogenous nucleic acids, to obtain the extracellular vesicles with high levels of the exogenous nucleic acids for diagnosis and treatment.

In this embodiment, the step of purifying the extracellular vesicles loaded with the exogenous nucleic acids, to obtain the extracellular vesicles with high levels of the exogenous nucleic acids for diagnosis and treatment, includes the following steps:

Purifying the extracellular vesicles loaded with the exogenous nucleic acids by using at least one of an ultrafiltration method and an RNase method to remove free nucleic acids, thereby obtaining the extracellular vesicles with high levels of the exogenous nucleic acids for diagnosis and treatment.

S103, quantifying the exogenous nucleic acids of the extracellular vesicles with high levels of the exogenous nucleic acids for diagnosis and treatment.

In this embodiment, the exogenous nucleic acids of the extracellular vesicles with high levels of the exogenous nucleic acids for diagnosis and treatment are quantified by at least one of real-time quantitative polymerase chain reaction (qPCR) and droplet digital polymerase chain reaction (ddPCR).

It can be understood that, by quantifying the exogenous nucleic acids in the extracellular vesicle, the quantitative exogenous nucleic acids enter the target cells and exert function thereof.

The present disclosure also provides a pharmaceutical composition, including the above-mentioned extracellular vesicles. By treating the target cells with the extracellular vesicles with high levels of the exogenous nucleic acids for diagnosis and treatment, the above-mentioned pharmaceutical composition transports the target nucleic acids into the target cells, corrects erroneous gene expressions in the target cells, enables integrated diagnosis, treatment, and theragnosis, thereby exerting diagnosis and treatment effects.

The extracellular vesicle provided by the disclosure can be used in regulating protein expression, protein replacement therapy, immunodiagnosis and immunotherapy, or tissue regeneration.

In the above-mentioned embodiments, the disclosure loads the therapeutic and diagnostic exogenous nucleic acids into the extracellular vesicles through the high-throughput physical squeezing method. Since a feature size of the nanoporation device (ExoNP) is close to or slightly smaller than that of the extracellular vesicles, a squeezing effect induced by geometric confinement is exerted on the extracellular vesicles, causing the membrane of each of the extracellular vesicle to undergo transient poration, thereby loading the exogenous nucleic acids therein, and protecting the in-vitro stability of the nucleic acid. Furthermore, by quantitatively designing the exogenous nucleic acids of the extracellular vesicles with high levels of the exogenous nucleic acids for diagnosis and treatment, complicated and uncontrollable biochemical processes are avoided. The quantitative exogenous nucleic acids enter target cells and exert functions thereof to precisely regulate protein expression, thereby increasing a utilization rate of the exogenous nucleic acids. The application of the pharmaceutical composition including the extracellular vesicles with high levels of the exogenous nucleic acids for diagnosis and treatment can serve as a supplement to or even replacement for novel diagnostic and treatment methods such as gene diagnosis and treatment and protein diagnosis and treatment.

Combining with the embodiments, the above-mentioned technical solutions are further described in detail hereinafter.

Using the above-mentioned rapid preparation method, changes were made to parameters, as shown in the table below.

| | Embodiment 1 | Embodiment 2 | Embodiment 3 | Embodiment 4 |
|---|---|---|---|---|
| Nucleic acid length | 21 | 1000 | 2000 | 3000 |
| (nucleotides) | | | | |
| Nucleic acid | 1 nM | 1 μM | 1 μM | 1 μM |
| concentration | | | | |
| Vesicle size (nm) | 150 | 150 | 150 | 150 |
| Vesicle | 1 × 10⁹ | 1 × 10¹⁰ | 1 × 10¹¹ | 1 × 10¹¹ |
| (particles/mL) | | | | |
| Method | Physical | Physical | Physical | Physical |
| | squeezing | squeezing | squeezing | squeezing |
| | method | method | method | method |
| Small molecule | — | — | — | — |
| Ratio (nucleic | 1:200 | 1:200 | 1:200 | 1:200 |
| acid:vesicle) | | | | |
| Loading efficiency | 10-fold | 8-fold | 5-fold | 2-fold |
| | enrichment | enrichment | enrichment | enrichment |
| Gene regulation | 2.5-fold | 2-fold | 2-fold | 2-fold |
| | downregulation | downregulation | downregulation | downregulation |

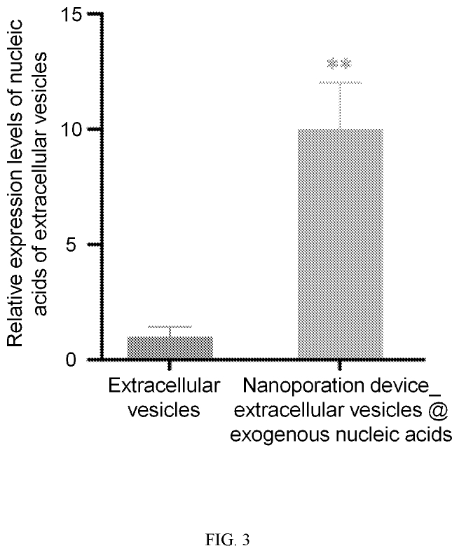

FIG. 3 is a graph showing a result of relative expression levels of nucleic acids in the extracellular vesicles of different treatment groups provided in embodiment 1. As shown in FIG. 3, compared to the “extracellular vesicles” group, the extracellular vesicles treated by the physical squeezing method using the nanoporation device achieve a 10-fold enrichment of the therapeutic and diagnostic exogenous nucleic acids.

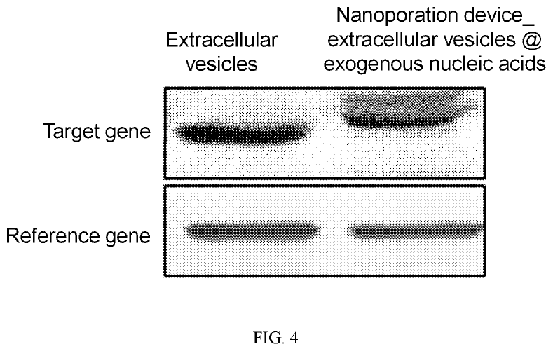

FIG. 4 is a graph showing a result of regulation of target genes in target cells by different treatment groups provided in embodiment 1. As shown in FIG. 4, compared to the “extracellular vesicles” group, the “nanoporation device_extracellular vesicles @ exogenous nucleic acids” group plays a role in down-regulating the target gene.

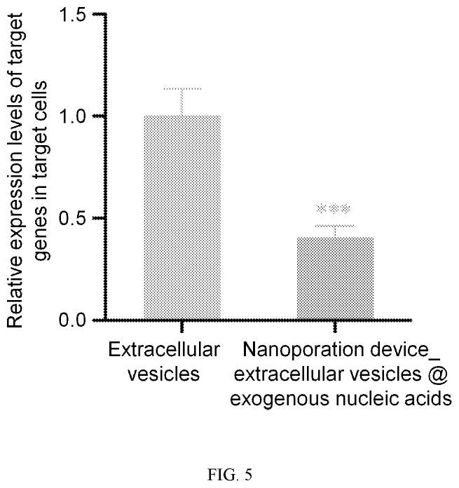

FIG. 5 is a graph showing a result of relative expression levels of target genes in target cells of different treatment groups provided in embodiment 1. As shown in FIG. 5, compared to the “extracellular vesicles” group, the “nanoporation device_extracellular vesicles @ exogenous nucleic acids” group achieves a 2.5-fold downregulation of the target gene.

As shown in the above-mentioned embodiment, the extracellular vesicles with high levels of the exogenous nucleic acids for diagnosis and treatment provided by the disclosure can achieve stable and controllable loading of the exogenous nucleic acid into the extracellular vesicles, and transport and release the quantitative exogenous nucleic acids into target cells to take effects, thereby increasing a utilization rate of the exogenous nucleic acids.

It can be understood that, the features of the above-mentioned embodiments can be combined in any way. To keep the description concise, not all possible combinations of the technical features in the above-mentioned embodiments are described. However, only if there is no contradiction in the combination of these technical features, they should be considered as falling within the scope of this description.

The above descriptions are only preferred embodiments of the present disclosure and only specifically describe the technical principles of the present disclosure. These descriptions are only for explaining the principles of the present disclosure and should not be construed as limiting the protection scope of the present disclosure in any way. Based on the explanations herein, any modifications, equivalent substitutions and improvements made within the spirit and principles of the present disclosure, as well as other specific embodiments of the present disclosure that can be conceived by those skilled in the art without inventive effort, should all be included within the protection scope of the present disclosure.

What is claimed is:

1. A method for preparing extracellular vesicles, characterized in that, the method comprises the following steps:
generating squeezing effects on extracellular vesicles, to load exogenous nucleic acids into the extracellular vesicles, wherein a concentration of the extracellular vesicles is 1×10²-1×10²⁰ particles/mL;
purifying the extracellular vesicles loaded with the exogenous nucleic acids, to obtain the extracellular vesicles with high levels of the exogenous nucleic acids for diagnosis and treatment; and
quantifying the exogenous nucleic acids of the extracellular vesicles with high levels of the exogenous nucleic acids for diagnosis and treatment.

2. The method for preparing extracellular vesicles according to claim 1, characterized in that, the step of generating squeezing effects on extracellular vesicles, to load exogenous nucleic acids into the extracellular vesicles, comprises the following steps:
mixing the exogenous nucleic acids with the extracellular vesicles, and then generating the squeezing effects on the extracellular vesicles via geometric constriction within a nanoporation device, inducing transient membrane poration of each of the extracellular vesicles, thereby loading the exogenous nucleic acids into the extracellular vesicles.

3. The method for preparing extracellular vesicles according to claim 1, characterized in that, a size of each of the extracellular vesicles is 20 nm-12 μm, and each of the exogenous nucleic acids comprises from 4 to 30000 nucleotides.

4. The method for preparing extracellular vesicles according to claim 3, characterized in that, the size of each of the extracellular vesicles is 30 nm-200 nm, and each of the exogenous nucleic acids comprise from 10 to 50 nucleotides.

5. The method for preparing extracellular vesicles according to claim 3, characterized in that, a concentration of the exogenous nucleic acids is 10 pM-1 μM, a concentration of the extracellular vesicles is 1×10⁴-1×10¹⁸ particles/mL, and a volume ratio of the exogenous nucleic acids to the extracellular vesicles is 1:50-1:500.

6. The method for preparing extracellular vesicles according to claim 5, characterized in that, the concentration of the exogenous nucleic acids is 1 nM-1 μM, and the volume ratio of the exogenous nucleic acids to the extracellular vesicles is 1:100-1:200.

7. The method for preparing extracellular vesicles according to claim 1, characterized in that, the step of purifying the extracellular vesicles loaded with the exogenous nucleic acids, to obtain the extracellular vesicles with high levels of the exogenous nucleic acids for diagnosis and treatment, comprises the following steps:
purifying the extracellular vesicles loaded with the exogenous nucleic acids by using at least one of an ultrafiltration method and an RNase method to remove free nucleic acids, thereby obtaining the extracellular vesicles with high levels of the exogenous nucleic acids for diagnosis and treatment.

8. The method for preparing extracellular vesicles according to claim 1, characterized in that, the step of quantifying the exogenous nucleic acids of the extracellular vesicles with high levels of the exogenous nucleic acids for diagnosis and treatment, comprises the following steps:
quantifying the exogenous nucleic acids of the extracellular vesicles with high levels of the exogenous nucleic acids for diagnosis and treatment, by at least one of real-time quantitative polymerase chain reaction and droplet digital polymerase chain reaction.

9. The method for preparing extracellular vesicles according to claim 1, characterized in that, the extracellular vesicles comprise liposomes, artificial lipid vesicles, lipid nanoparticles, plant extracellular vesicles, animal extracellular vesicles, human cell-derived extracellular vesicles, bacterial outer membrane vesicles, microbial secreted membrane vesicles, cell membrane reassembled nanoparticles, bacterial outer membrane reassembled nanoparticles, enveloped virus particles and virus-like particles, and artificial nanovesicles hybridized with cell membrane and/or bacterial outer membrane and/or viral membrane protein; the exogenous nucleic acids for diagnosis and treatment comprise plasmids, ribonucleic acids, deoxyribonucleic acids, oligonucleotides, circular RNA, miRNA, mRNA, antisense oligonucleotides (ASO), siRNA, saRNA, splice-switching oligonucleotide (SSO), and expressed oligonucleotides (EON).

10. A pharmaceutical composition, comprising extracellular vesicles prepared by the method according to claim 1.

11. Use of the pharmaceutical composition according to claim 10 in regulating protein expression, protein replacement therapy, immunodiagnosis and treatment, or tissue regeneration.
